(19) United States
(12) Patent Application Publication
(10) Pub. No.: US 2005/0158271 A1
(43) Pub. Date: Jul. 21, 2005

(54) Pharmaceutical applications of hydrotropic polymer micelles

(76) Inventors: Sang Cheon Lee, Seoul (KR); Kang Moo Huh, Taejeon (KR); Jae Hyung Park, West Lafayette, IN (US); Kinam Park, West Lafayette, IN (US)

Correspondence Address: JAMES H. MEADOWS AND MEDICUS ASSOCIATES, 2804 KENTUCKY, JOPLIN, MO, 64804, US

(21) Appl. No.: 10/961,831

(22) Filed: Oct. 9, 2004

Related U.S. Application Data
(63) Continuation-in-part of application No. 09/975,800, filed on Oct. 11, 2001.
(60) Provisional application No. 60/239,455, filed on Oct. 11, 2000.
(60) Provisional application No. 60/294,957, filed on May 31, 2001.

(51) Int. Cl. A61K 31/785

(52) U.S. Cl. 424/78.3

(57) ABSTRACT

Hydrotropic polymer micelles effective for increasing the water solubility of poorly soluble drugs are described. Such hydrotropic polymer micelles have the combined properties of polymer micelles and hydrotropic agents, which display a synergistic effect for increasing the solubility of such drugs. Hydrotropic polymer micelles are formed in solution from amphiphilic copolymers that comprise a hydrophilic polymer and a hydrophobic polymer having pendant hydrotropic agents. A preferred copolymer is a di-, tri- or multi-block copolymer composed of hydrophilic and hydrophobic polymer chains. A particularly preferred hydrophilic chain comprises polyethyleneoxide (PEG) and a preferred hydrophobic chain comprises hydrotropic monomer units derived from nicotinamide. The micelles are found to be much more effective in solubilizing poorly soluble drugs and exhibit an excellent long-term stability even at high loading of drugs. A hydrotropic polymer micelle has nanometer scale size, by which they can deliver poorly soluble drugs to the body through diverse routes of administration.

### CROSS-REFERENCE TO RELATED APPLICATIONS

The present application is a continuation-in-part of U.S. Ser. No. 09/975,800, filed Oct. 11, 2001, which claims the benefit of priority of U.S. Provisional No. 60/239,455, filed Oct. 11, 2000, and of U.S. Provisional No. 60/294,957, filed May 31, 2001. The disclosures of the aforementioned patent applications are incorporated herein by reference.

### FIELD OF THE INVENTION

The present invention relates to chemical compositions and methods of drug delivery, particularly those relating to delivery of poorly soluble drugs.

### BACKGROUND OF THE INVENTION

Many drugs and drug candidates are poorly water soluble, and such poor solubility causes significant problems in drug development, formulation, and absorption.(1, 2) To date, various solubilizing systems have been explored to improve the bioavailabilities of poorly soluble drugs by enhancing water solubilities.(3-5)

Paclitaxel is a good model drug for describing the significance of those factors affecting delivery of poorly soluble drugs. Application of paclitaxel in cancer therapy has been limited by its extremely low water solubility (0.3 μg/ml).(6) Currently, paclitaxel is dissolved in a 50:50 mixture of Cremophore EL and dehydrated ethanol, which is further diluted in isotonic saline solution before intravenous (i.v.) administration.(7) The paclitaxel solubility in this formulation does not exceed 1.2 mg/mL.(8) Thus, large volumes of formulations need to be delivered to obtain recommended paclitaxel doses of 135 mg/m² and 175 mg/m² for small (1.4 m²) and large (2.4 m²) patients.(8) In addition, the diluted clinical formulation has only short-term physical stability (12˜24 h), and tends to precipitate from the aqueous media.(9) For this reason, there is a growing need to develop alternative solubilizing systems with a high solubility-enhancing capacity as well as a good long-term physical stability.

The poor bioavailability of poorly water-soluble drugs becomes even worse when the drug is given orally.(10) Since oral administration is the most convenient method of delivering drugs and is used for the majority of drugs, developing a method for increasing the water-solubility of poorly soluble drugs is highly important. Increasing the water-solubility of poorly water-soluble drugs should allow development of effective oral dosage forms. Dissolution of the active ingredient from a conventional dosage form (e.g., tablet or suspension) is one of the most critical steps in drug absorption leading to bioavailability. For poorly water-soluble drugs, dissolution in aqueous media is often the primary limitation. When the aqueous solubility of a drug is smaller than 0.1 mg/ml, dissolution of the drug is too slow for effective absorption of the drug.(11) Moreover, systemic delivery of paclitaxel in large doses is limited by hematologic toxicity, neutropenia, and dose-dependent neurotoxicity. The ability to deliver a smaller amount of paclitaxel by oral administration may reduce the toxicity associated with large doses given i.v. every few weeks, since oral administration generally enjoys better compliance.

An increase in the water-solubility of poorly soluble drugs should provide new avenues of drug delivery that have not been possible before. Thus, there has been much effort directed to the development of a diverse class of solubilizing systems including liposomes, cyclodextrins, emulsions, mixed-micelles, microspheres, and polymeric micelles. Existing approaches for improving the water-solubility of poorly soluble drugs include the following: (1) synthesis of prodrugs and analogs; (2) physical modification of drugs; (3) use of cosolvents; (4) emulsions, micelles, and liposomes; (5) complexation; (6) solid dispersion technology; and (7) use of hydrotropic agents (hydrotropes).

Among various carrier systems, polymeric micelles derived from amphiphilic block copolymers have been widely pursued for a wide variety of poorly soluble drugs.(12-15) In an aqueous phase, the hydrophobic block of the copolymer forms the inner core of the micelles while the hydrophilic block forms the outer shell. The inner core serves as a microenvironment for solubilization of poorly soluble drugs. The high potential of polymeric micelles as a drug carrier lies in their unique characteristics, such as nano-size and thermodynamic stability. In addition, their core-shell structure can mimic naturally occurring transport systems such as plasma lipoproteins and viruses, satisfying the structural aspect to act as a transport system in a body.

To date, two leading groups, Kabanov's and Kataoka's, have made great contributions to this field. Kabanov's work initially focused on micelles constructed from PEO-b-PPO-b-PEO (Pluronics) triblock copolymers as a drug carrier across the blood brain barrier.(13) The focus of Kataoka's group was the micelles formed from amphiphilic copolymers containing a poly(amino acid) core-forming block, as delivery vehicles for anti-cancer drugs.(14, 15) The incorporation of drug molecules into the inner core of micelles is achieved by chemical conjugation of drugs to the core-forming block or by physical interaction of drugs with the micellar core. In recent years, the physical entrapment of the poorly soluble drugs inside micelles is much more preferred due to the maintenance of drug activity inside micelles without the need for chemical modification.

Most previous polymeric micelles are based on hydrophilic poly(ethylene glycol) (PEG) since PEG is a biocompatible polymer that expresses low toxicity and, when located at the surface and interface, suppresses protein and cellular adsorption. Thus, the structural variation has been made mainly with the hydrophobic block such as aliphatic polyesters, poly(amino acids) and poly(propylene oxide). Table 1 lists some previous examples of PEG-containing block copolymers for solubilization of drugs.

TABLE 1
Amphiphilic block copolymers for a micellar carrier of drugs

| Block copolymers | Drugs | Reference |
|---|---|---|
| poly(ethylene oxide)-b- | doxorubicin | Kwon et al. (15) |
| poly(β-benzyl L-aspartate) | | Kataoka et al. (14) |
| poly(ethylene oxide)-b- | haloperidol | Kabanov et al. (13) |
| poly(propylene oxide) | | |
| poly(ethylene oxide)-b- | dihydrotestosteron | Eisenberg et al. (16) |
| poly(ε-caprolactone) | paclitaxel | Kim et al. (17) |
| poly(acrylic acid)-b- | doxorubicin | Hoffman et al. (18) |
| oligo(methyl methacrylate) | | |
| poly(ethylene oxide)-b- | paclitaxel | Burt et al. (19) |
| Poly(D,L-lactide) | | Kim et al. (20) |
| Poly(2-ethyl-2-oxazoline)- | paclitaxel | Lee et al. (21) |
| b- poly(ε-caprolactone) | | |

Although polymeric micelles based on previous amphiphilic block copolymers have shown high potentials as drug solubilizing systems, most polymeric micelles have shown limited solubilizing capacity for paclitaxel, and, in most cases, maximum contents of paclitaxel loaded in micelles was around 20 wt %.(17, 19, 21) Besides, a simple polymer design may not effectively predict whether the resulting polymer micelles show high solubilizing capacity. A more serious limitation is the poor stability of paclitaxel-solubilized polymeric micelles in water, and the stability tends to become lower as the content of paclitaxel increases.(19)

The term “hydrotropy” refers to a solubilization process whereby the addition of large amounts of a second solute results in an increase in the aqueous solubility of a poorly soluble compound.(22) Hydrotropic agents (or hydrotropes) are compounds that, at high concentrations, solubilize poorly water-soluble molecules in water.(23) At concentrations higher than a minimal hydrotrope concentration, hydrotropic agents self-associate and form noncovalent assemblies of lowered polarity, i.e., nonpolar microdomains, which solubilize hydrophobic solutes.(24) The self-aggregation of hydrotropic agents is different from surfactant self-assemblies (i.e., micelles) in that hydrotropes form planar or open-layer structures instead of compact spheroid assemblies.(25) Hydrotropic agents are structurally characterized by having a short, bulky, compact moiety, such as an aromatic ring, while surfactants are characterized by long hydrocarbon chains. In general, hydrotropic agents have a shorter hydrophobic segment, leading to higher water solubility, than do surfactants. Hydrotropy is suggested to be superior to other solubilization methods, such as micellar solubilization, miscibility, cosolvency, and salting-in, because the solvent character is independent of pH, has high selectivity, and does not require emulsification.(26)

Examples of hydrotropic materials used as excipients in the literature are sodium salicylate, sodium gentisate, sodium glycinate, nicotinamide, sodium benzoate, sodium toluate, sodium ibuprofen, pheniramine, lysine, tryptophan, and isoniazid.(23) Each hydrotropic agent is effective in increasing the water solubility of selected hydrophobic drugs; no universal hydrotropic agent has been found effective to solubilize all hydrophobic drugs. Thus, finding the right hydrotropic agents for a poorly soluble drug requires screening a large number of candidate hydrotropes. However, once the effective hydrotropic agents are identified for a series of structurally different drugs, the structure-activity relationship can be established.

Of the various approaches listed above, the hydrotrope approach is a highly promising new method with great potential for poorly soluble drugs, in general. For instance, should the solubility of paclitaxel be increased by 2-4 orders of magnitude in the presence of hydrotropic compounds, the oral absorption and subsequent bioavailability is also expected to increase by a similar extent. The increase in solubility is also expected to be beneficial in overcoming the adverse effects of P-glycoproteins in the GI tract, due to excess drug saturating the P-glycoproteins. This consideration is especially important for those conditions that are largely untreatable due to multi-drug resistance, e.g., certain breast cancers.

Using hydrotropic agents is one of the easiest ways of increasing water-solubility of poorly soluble drugs, since it only requires mixing the drugs with the hydrotrope in water. The hydrotrope approach does not require chemical modification of hydrophobic drugs, use of organic solvents, or preparation of emulsion systems. Despite these advantages, hydrotropes have not been widely explored for increasing the water solubility of poorly soluble drugs. The main reason for this may be a concern that the use of low molecular weight hydrotropic agents may result in the co-absorption of a significant amount of the hydrotropic agent either from the GI tract after oral administration or from the bloodstream after parenteral injection.

Previously, the synthesis of polymers based on polymerizable derivatives of 5-oxo-pyrrolidinecarboxylic acid and pyrrolidonyl oxazoline monomers has been reported. (U.S. Pat. Nos. 4,933,463; 4,981,974 and 5,008,367 to Dandreaux etal.; U.S. Pat. Nos. 4,946,967 and 4,987,210 to Login et al.) The structures of the aforementioned polymers are modifications of polyvinylpyrrolidone (PVP), a well-known synthetic polymer having a variety of applications. Steric crowding between the hydrophilic pyrrolidone ring and hydrophobic hydrocarbon backbone of the PVP polymer was proposed to limit complexation of the polymer with other molecules, especially when dipole-dipole interactions are involved. (Dandreaux et al.). Accordingly, the investigators synthesized pyrrolidone-containing polymers wherein the pyrrolidone ring is spaced away from the polymer backbone. The resulting polymers reportedly show an increase in water solubility of selected organic compounds. Since the structures of these polymers are based on PVP, the range of compounds is very limited. Moreover, the aforementioned PVP-based polymers are not believed to be particularly water-soluble and, therefore, are not expected to display pronounced hydrotropic properties.

Additionally, a class of amphiphilic block copolymers has been described, which are based on polymerizable derivatives of pyridine. See, e.g., U.S. Pat. Nos. 6,383,500 (to Wooley et al.) and 6,491,903 (to Forster et al.), and related patents and patent publications. Reportedly, the amphiphilic copolymers form micelles in water, which are crosslinked in the shell domain, and optionally crosslinked in the core domain.

Another class of hydrophilic polymeric compounds, e.g., represented by PEGs and water-soluble carbohydrates, reportedly has been studied for the ability to increase water solubility of certain structurally similar drugs, particularly quinazoline-, nitrothiazole-, and indolinone-based compounds. (U.S. Pat. No. 6,248,771 to Shenoy et al.) The PEGs used in the formulations of this reference are provided as surfactants for the drug compound. The combination of a pharmacologically active compound, such as cyclosporin, with a monoester made from a fatty acid and a polyol, such as a saccharide, also has been proposed. (U.S. Pat. No. 5,756,450 to Hahn et al.) The use of peptides, such as gelatins, in formulations to increase the solubility of the drug has been suggested. (U.S. Pat. No. 5,902,606 to Wunderlich et al.)

An object of the present invention is to provide a new class of hydrotropic polymer micelles that permit high loading of poorly soluble drugs therein. Another object of the invention is to develop hydrotropic polymer micelles that have prolonged stability in aqueous solution, particularly when loaded with high levels of a poorly soluble drug. Paclitaxel is an advantageous model drug compound for testing such micelles.

### SUMMARY OF THE INVENTION

The present invention is for novel compositions of matter and methods employing hydrotropic polymer micelles as excipients to increase the aqueous solubility of poorly soluble drugs. The present invention employs novel block, graft, and random copolymers consisting of hydrophilic monomer units and hydrophobic monomer units, which possess hydrotropic moieties. Paclitaxel is illustrated as a model poorly soluble drug.

A copolymer of the invention is effective for increasing aqueous solubility of a poorly soluble drug, which copolymer comprises a plurality of at least one hydrophilic monomer unit and a plurality of at least one hydrophobic monomer unit, wherein the hydrophobic monomer unit possesses a pendant hydrotropic moiety. The copolymer preferably has at least one hydrophilic monomer is selected from the group consisting of ethylene glycol, oligoethylene glycol methacrylate, acrylic acid, methacrylic acid, N-isopropylacrylamide, N-vinylpyrrolidone, 2-methyl-2-oxazoline, and 2-ethyl-2-oxazoline. The copolymer preferably has a plurality of hydrophilic monomer units is present in the copolymer as a hydrophilic polymer block. The copolymer preferably has at least one hydrophobic monomer unit is selected from the group consisting of polymerizable derivatives of nicotinamide or salicylate, more preferably the hydrophobic monomer unit is an acryl or styryl derivative of nicotinamide or salicylate.

In another aspect, a pharmaceutical composition comprises a plurality of hydrotropic polymer micelles loaded with a pharmacologically effective amount of a poorly soluble drug, wherein the micelles are comprised of an amphiphilic copolymer formed of a plurality of at least one hydrophilic monomer unit and a plurality of at least one hydrophobic monomer unit possessing a pendant hydrotropic moiety. Preferably, the composition has a plurality of at least one hydrophilic monomer unit present in the copolymer in the form of poly(ethylene glycol), poly(oligoethylene glycol methacrylate), poly(acrylic acid), poly(methacrylic acid), poly(N-isopropylacrylamide), poly(N-vinylpyrrolidone), poly(2-methyl-2-oxazoline), or poly(2-ethyl-2-oxazoline). Preferably, the composition has a plurality of at least one hydrophobic monomer unit present in the copolymer in the form of a block of polymerizable derivatives of nicotinamide and N-substituted nicotinamide.

Also contemplated is a method of treating a patient with a drug comprising co-administering the drug and a hydrotropic polymer micelle to the patient.

### BRIEF DESCRIPTION OF THE DRAWINGS

FIG. 1 illustrates schematically the formation of hydrotropic polymer micelles from a diblock copolymer according to the principles of the present invention.

FIG. 2 depicts paclitaxel loading contents in PEG₅₀₀₀-b-P(VBODENA)₄₃₅₀ micelles dissolved in acetonitrile or N,N-dimethylacetamide (DMAc) as a function of the feed weight ratio of polymer to paclitaxel.(n=3) The filled triangle represents the maximum loading content of paclitaxel in PEG₂₀₀₀-b-PDLLA₂₀₀₀ micelles as a control. PDLLA=Poly(D,L-lactide).

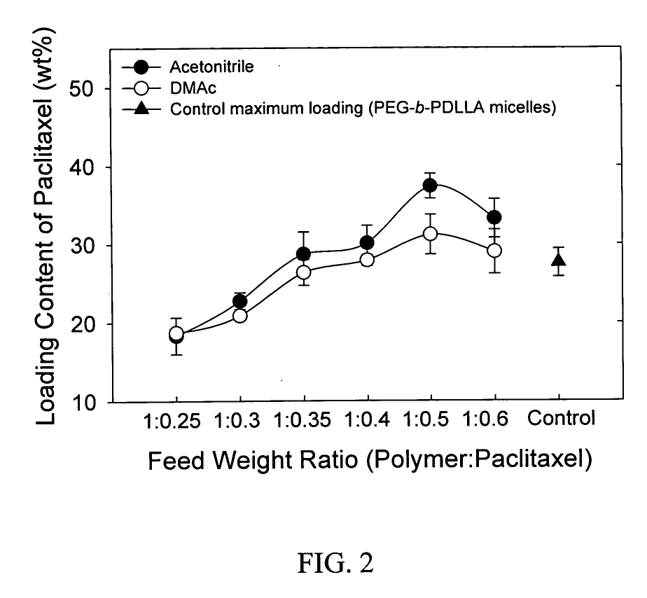

FIG. 3 shows stability of paclitaxel-loaded micelles formed from PEG₅₀₀₀-b-P(VBODENA)₄₃₅₀ copolymer in water. The percent changes in the initial paclitaxel concentration loaded into the micelles are shown.(n=3)

### DETAILED DESCRIPTION OF THE INVENTION

The present invention affords efficient compounds and methods for increasing the solubility of a diverse class of poorly water-soluble drugs. As used herein, the term “poorly soluble drugs,” and equivalents thereof, refers to pharmaceutical compounds having a water solubility of less than about 100 μg/ml at 37° C. Representative examples are paclitaxel, griseofunvin, progesterone, and tamoxifen.

The present invention affords a polymer micelle, referred to herein as a “hydrotropic polymer micelle,” and equivalents thereof, which can increase the inherent aqueous solubility of a target drug. A hydrotropic polymer micelle is formed by polymeric molecules that self-aggregate in a suitable solvent. Preferably, the polymer molecules are block copolymers of a hydrophilic polymer and a hydrophobic polymer that contains at least one hydrotropic moiety.

Without wishing to be limited to any particular theory, it is believed that a hydrotropic polymer micelle of the present invention increases the water solubility of a poorly soluble drug by a synergistic effect of micellar and hydrotropic solubilization. Such a hydrotropic polymer micelle shows superiority to other currently used polymer micelles in terms of solubilizing capacity and long-term stability.

Many candidate hydrotropic agents (or hydrotropes) were tested for their ability to increase aqueous paclitaxel solubility as a model for and proof of concept for the present invention. A number of nicotinamide analogs were synthesized based on the observation that nicotinamide showed a good hydrotropic property. N,N-Diethylnicotinamide (DENA) was found to be the most effective hydrotropic agent for paclitaxel of those studied. The aqueous paclitaxel solubility was 39 mg/ml and 512 mg/ml at the DENA concentrations of 3.5 M and 5.95 M, respectively.(27) These values represent a 5˜6 orders of magnitude increase in aqueous solubility over the intrinsic solubility of 0.3 μg/ml. N-Picolylnicotinamide (PNA), N-allylnicotinamide, and sodium salicylate were also excellent hydrotropes for paclitaxel. The molecular structures of these hydrotropic agents are shown below. The water solubility data showed that an effective hydrotropic agent should be highly water soluble while maintaining a hydrophobic segment.

TABLE 2
Chemical structures of identified hydrotopes for paclitaxel

| N,N-Diethylnicotin- | N-Picolylnicotinamide | | Sodium |
|---|---|---|---|
| amide (DENA) | (PNA) | N-Allylnicotinamide | salicylate |
| 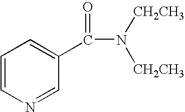 | 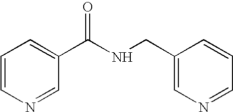 | 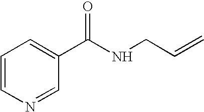 | 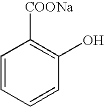 |

Recently, polymers and hydrogels based on effective hydrotropic agents such as DENA and PNA were synthesized to develop new polymeric solubilizing systems that maintain the benefits of hydrotropy.(28) The hydrotropic property of the hydrotropes was maintained in their polymeric forms, and a highly localized concentration of the hydrotrope in polymers and hydrogels was found to be a main contributor to effective solubilization of paclitaxel.

Of the properties of solubilizing systems, a high solubilizing capacity and a good physical stability are the two most important factors in determining whether a drug delivery system is clinically useful or not. Thus, it is desired to develop a hydrotropic polymeric micelle having a high solubilizing capacity for poorly water-soluble drugs as well as a good long-term stability. An approach taken here is to incorporate hydrotropic properties into the micellar inner core by preparing polymeric micelles from amphiphilic diblock copolymers consisting of hydrophilic PEG and a hydrophobic polymer containing hydrotropic moieties. In water, the amphiphilic block copolymers are expected to self-associate to form micelles of the PEG outer shell and the hydrotrope-rich inner core. Since an identified hydrotrope for a specific drug is introduced as a core component in a highly localized way, paclitaxel solubilization may be presented by the synergistic effect of both hydrotropic and micellar solubilization. The typically poor colloidal stability of previous micelles is believed caused by the enhanced hydrophobicity of micelles after solubilization of paclitaxel. Hence, hydrotropic moieties characterized by a strong hydrophilic nature are expected to permit good stability for paclitaxel-loaded micelles in water.

Accordingly, an aspect of the present invention is a hydrotropic polymer micelle, which is a self-assembly of amphiphilic copolymers consisting of hydrophilic polymers, such as PEG, poly(acrylic acid) (PAA), or poly(N-isopropylacrylamide) (PNIPAm), and a hydrophobic poly(meth)acrylate, which bears pendant (dangling) hydrotropic agents. A hydrotropic micelle of the invention comprises a hydrophilic outer shell and a hydrotrope-rich inner core in aqueous media. The polymer micelles are constructed by the assembly of about 200-300 polymer chains.(29) Normally, the micelle size range is 20-100 nm, and the dimension of the micellar core is smaller.(l 2) This assembly process leads to the localization of hydrotropic moieties within the limited core space of micelles, which results in the maximized concentration of hydrotropic moieties in a specific volume. Thus, the hydrotropic polymer micelles are expected to offer very high drug solubilities, since the localization of hydrotropic agents is much more pronounced than for the low molecular hydrotropes, the hydrotropic polymer and hydrogels. Besides, due to the nanoparticle properties, the viscosity problem observed in high concentration solutions of the linear hydrotropic polymers can also be circumvented.

The present invention illustrates the superiority of hydrotropic polymer micelles to current polymeric micelles and shows how the hydrotropic polymer micelles are different from existing systems. These questions can be answered by comparison in terms of the drug loading and the physical stability. The loading capacity of the normal polymer micelles for poorly soluble drugs is decided by various factors such as the length of the core-forming polymer and compatibility between drugs and the core-forming polymers.(12) Of these factors, compatibility is the most significant in determining the solubilizing capacity. One parameter, which has been used to assess the compatibility between solubilizates and the polymer, is the Flory-Huggins interaction parameter. This value is dependent on a pair of the selected drug and the polymer. Due to the uniqueness of each drug, no one core-forming block can maximize the solubilization level for all drugs. Thus, to find or synthesize the right structure of the polymer for effective solubilization is the first priority. However, the number of biocompatible polymers is limited and requires synthesis, which is inevitable to screen a large number of the polymer structures for effective solubilization of a selected drug. On the other hand, the hydrotropic approach is simple and much less laborious, even though the screening process is also required. Many hydrotropes can be identified for a selected drug by a simple mixing procedure, and the broad range of the chemical structures can be readily screened.(27) The key concept of the hydrotropic polymer micelles is based on the hydrotrope-containing core-forming polymers. Thus, systematic design affords more efficient systems for solubilizing poorly soluble drugs.

Another advantage expected from this unique approach is the enhanced physical stability of the formulations. This property is one measure that makes the hydrotropic polymer micelles distinguished from normal polymer micelles. Conventional polymer micelles have a core with a strong hydrophobic nature, namely, the drug solubilization has been expected only by the hydrophobic interaction between drugs and the inner core. Thus, it is often the case that the polymer micelles loaded with drugs cannot overcome the enhanced hydrophobicity and the secondary aggregation between micelles, resulting in the precipitation of rug in water. In this point of view, the hydrotropic polymer micelles provide formulations with a good stability even at a high loading of poorly soluble drugs due to the hydrophilic nature of the hydrotropes that reside in the micellar core domains. Of course, the same approach can be used for solubilization of other poorly soluble drugs. The availability of the new hydrotropic polymer micelles of the present invention permits development of novel delivery systems for many drugs and drug candidates of which applications have been limited previously due to their poor water solubilities.

### I. Hydrotropic Polymer Micelles

Although many hydrotropic agents are considered safe and some have been used in humans, the use of rather high concentrations of the hydrotropic agents may pose a difficulty in formulation of drug delivery systems. This is mainly due to the possibility of absorption of a low molecular weight hydrotropic agent itself from the dosage form into the body, such as from the GI tract into the bloodstream. Besides, newly synthesized hydrotropic polymers effective for increasing water solubility of poorly soluble drugs also have a drawback in producing useful dosage forms due to high viscosity and low physical stability.(28) For this reason, it is desirable to make a formulation, which has not only excellent physical stability but also a hydrotropic property capable of solubilizing a large amount of a poorly soluble drug. A hydrotropic polymer micelle, which has the combined properties of micellar and hydrotropic solubilization, promises an ideal system to overcome difficulties encountered with hydrotropic agents and hydrotropic polymers.

### A. Synthesis of Copolymers of PEG and Hydrotropic Polymers

Table 3 lists some of the block and graft copolymers consisting of poly(ethylene glycol) (PEG) and hydrotropic polymers that have been synthesized based on the molecular structures of identified hydrotropic agents for paclitaxel, such as N,N-diethylnicotinamide and N-picolylnicotinamide.

TABLE 3
Exemplary copolymers of PEG and hydrotropic polymers
synthesized from modified hydrotropic agents.

Poly(ethylene glycol)-block-poly(2-(4-vinylbenzyloxy)-N,N-
diethylnicotinamide)
Poly(ethylene glycol)-block-poly(2-(4-vinylbenzyloxy)-N-
picolylnicotinamide)
Poly(ethylene glycol)-block-poly(2-(4-vinylbenzyloxy)-nicotinamide)
Poly(oligoethylene glycol methacrylate-co-poly(2-(4-
vinylbenzyloxy)-N,N-diethylnicotinamide)
Poly(oligoethylene glycol methacrylate-co-poly(2-(4-vinylbenzyloxy)-N-
picolylnicotinamide)
Poly(oligoethylene glycol methacrylate-co-poly(2-(4-vinylbenzyloxy)-
nicotinamide)

The main components necessary for synthesis of the block copolymers are PEG modified with bromine or chlorine and a hydrotropic agent modified with polymerizable vinyl, acryl, or styryl groups. Table 4 lists some useful hydrotropic agents modified with an unsaturated double bond functionality, which permits polymerization. The relevant combination of monomers listed in Table 4 can lead to a diverse class of the copolymers capable of making the hydrotropic micelles.

TABLE 4
Modified hydrotropic monomers for synthesis of hydrotropic polymer micelles.

| 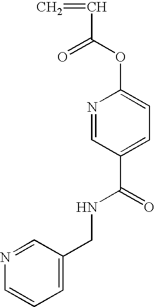 | 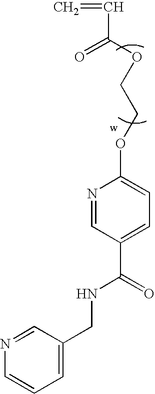 | 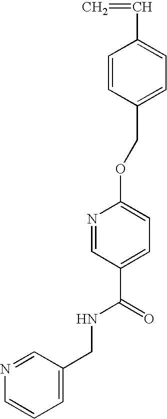 |
|---|---|---|
| A. 6- | B. 6-(2- | C. |
| acryloyl-N- | (acryloyl) | 6-(4-vinyl- |
| picolyl- | ethoxy- | benzyloxy)- |
| nicotinamide | ethoxy- | N-picolyl- |
| | ethoxy)-N- | N-picolyl- |
| | picolyl- | nicotinamide |
| | nicotinamide | |

| 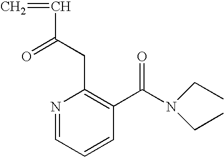 | 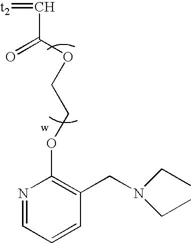 | 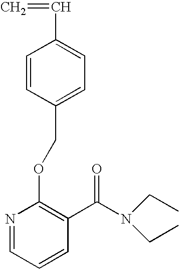 |
|---|---|---|
| D. | E. | F. |
| 2-acryloyl-N,N- | 2-(2-(acryloyl) | 2-(4- |
| diethyl- | ethoxyethoxy- | (vinylbenzyl |
| nicotinamide | ethoxy)-N,N-diethyl | oxy)-N,N- |
| | nicotinamide | diethyl- |
| | | nicotinamide |

The present invention is now discussed by way of certain examples, which illustrate but do not limit it.

### EXAMPLES

Unless otherwise noted, all reagents were purchased from Aldrich Chemical (Milwaukee, Wis.) or Sigma Chemical (St. Louis, Mo.).

### Example 1

### Synthesis of poly(ethylene glycol)-block-poly(2-(4-vinylbenzyloxy)-N,N-diethylnicotinamide)

An example of the synthesis of a block copolymer of the present invention is for poly(ethylene glycol)-block-poly(2-(4-vinylbenzyloxy)-N,N-diethylnicotinamide) as a model copolymer comprising a PEG block and a hydrotropic polymer block possessing N,N-diethylnicotinamide groups. The overall synthetic scheme for poly(ethylene glycol)-block-poly(2-(4-vinylbenzyloxy)-N,N-diethylnicotinamide) is shown below.

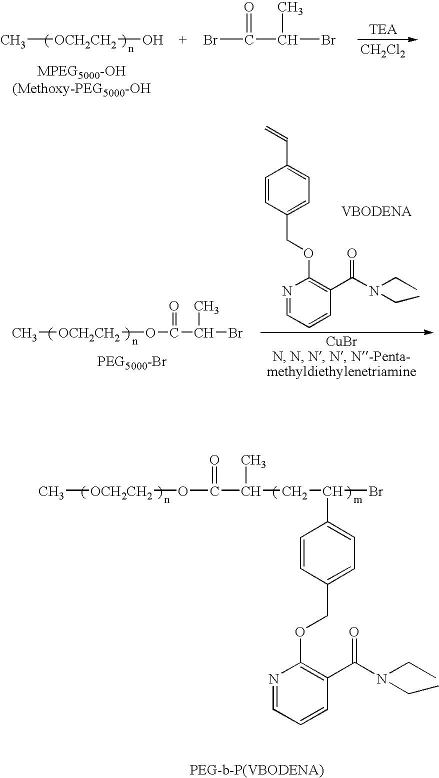

In the formula, n represents the number of oxyethylene units in the polymer, which is nominally 50000, and m represents the number of hydrotropic monomer units in the hydrotropic polymer, which is nominally 4350 in the example described hereinbelow.

### Example 2

### Synthesis of the PEG macroinitiator (PEG₅₀₀₀-Br)

A macroinitiator, PEG₅₀₀₀-Br, was synthesized as follows. A solution of PEG₅₀₀₀-OH (10 g, 2 mmol) and TEA (1.42 g, 14 mmol) in dry methylene chloride (50 mL) was placed into the flame-dried two-neck round-bottom flask equipped with a condenser, a dropping funnel, N₂ inlet/outlet, and a magnetic stirrer. After cooling to 0° C., 2-bromopropionyl bromide (BPB) (3.02 g, 14 mmol) in dry methylene chloride (10 mL) was then added dropwise to the stirred solution. The reaction mixture was stirred at room temperature under N₂ for 24 h. The crude reaction mixture was poured into cold diethyl ether, and the precipitates were filtered and washed with diethyl ether. The crude product was dissolved in methylene chloride (300 mL), and the solution was washed with distilled water (3×50 mL). The organic layer was dried over anhydrous magnesium sulfate and filtered. The PEG macroinitiator, PEG₅₀₀₀-Br, was then isolated by repeated precipitation from methylene chloride into cold diethyl ether. PEG₅₀₀₀-Br: Yield 82%.

### Example 3

### Synthesis of 2-(4-(vinylbenzyloxy)-N,N-diethylnicotinamide)) (VBODENA)

VBODENA was prepared by the reaction of 2-hydroxy-N,N-diethylnicotinamide (HDENA) with 4-vinylbenzyl chloride. 4-Vinylbenzyl chloride (5.89 g, 0.038 mol) was added dropwise to the suspension of HDENA (5 g, 0.026 mol) and potassium carbonate (7.12 g, 0.051 mol) in dry acetone (150 mL) at 70° C. The reaction mixture was stirred under nitrogen for 20 h. After the reaction, the crude reaction mixture was filtered, and the product was then isolated by column chromatography with THF/n-hexane on a silica gel. Further purification was performed by recrystallization from THF/n-hexane. Yield 90%.

### Example 4

### Synthesis of Diblock Copolymers of PEG and P(VBODENA) (PEG-b-P(VBODENA))

Synthesis of the block copolymers is described using PEG₅₀₀₀-b-P(VBODENA)₄₃₅₀ as an example. The PEG₅₀₀₀-Br macroinitiator (0.4 g, 0.08 mmol), VBODENA (0.366 g, 1.2 mmol), and Cu(I)Br (0.046 g, 0.32 mmol) were added to a flame-dried round-bottom flask. The flask was evacuated and refilled with dry nitrogen twice. Toluene (1.5 mL) was degassed separately and added into the flask. After the mixture was stirred and purged with N₂ for 10 min, N,N,N′,N′,N″-pentamethyl-diethylenetriamine (PMDETA) (0.054 g, 0.32 mmol) was introduced, and the flask was placed in a preheated oil bath. The reaction was maintained at 85° C. for 3 h. The reaction solution became gradually more viscous. After the polymerization, the reaction mixture was diluted with methylene chloride and passed through a silica gel column to remove the copper catalyst. The block copolymers were purified by repeated precipitation from methylene chloride into cold diethyl ether. Another block copolymer, PEG₅₀₀₀-b-P(VBODENA)₂₇₉₀ with different chain length of P(VBODENA), was synthesized in an identical manner except that a different feed molar ratio of VBODENA to EG unit of PEG₅₀₀₀-Br was employed. PEG₅₀₀₀-b-P(VBODENA)₄₃₅₀: Yield 91%.

### Example 5

### Synthesis of Methyl-poly(ethylene glycol)-block-poly(2-(4-vinylbenzyloxy)-N-picolylnicotinamide))

An example of the synthesis of a block copolymer is for poly(ethylene glycol)-block-poly(2-(4-vinylbenzyloxy)-N-picolylnicotinamide) as a model copolymer comprising a PEG block and a hydrotropic polymer block possessing N-picolylnicotinamide groups. The overall synthetic route for poly(ethylene glycol)-block-poly(2-(4-vinylbenzyloxy)-N-picolylnicotinamide)) is shown below.

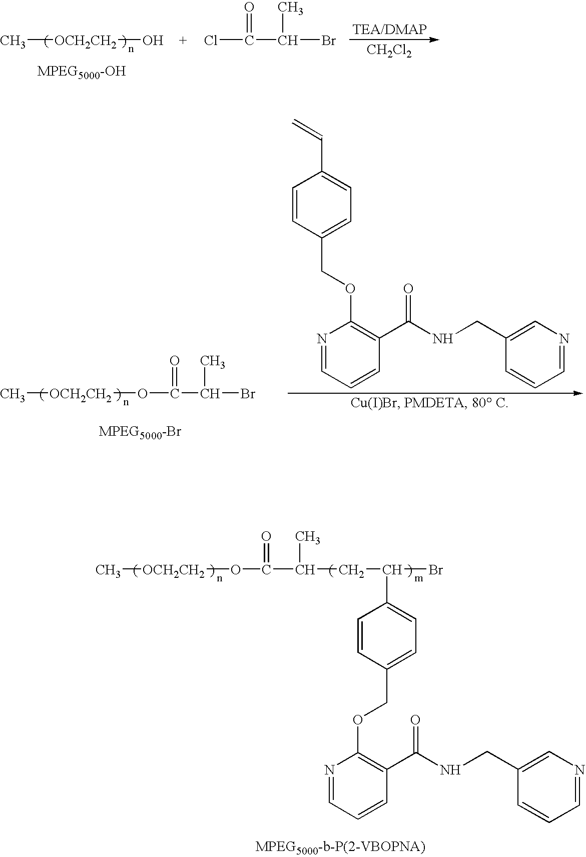

### Example 6

### Synthesis of 2-(4-(vinylbenzyloxy)-N-picolylnicotinamide) (2-VBOPNA)

2-VBOPNA was prepared by the reaction of 2-hydroxy-N-picolylnicotinamide (2-HPNA) with 4-vinylbenzyl chloride. In brief, 4-vinylbenzyl chloride (6.66 g, 0.044 mol) was added dropwise to a suspension of 2-HPNA (5 g, 0.022 mol) and K₂CO₃ (7.54 g, 0.055 mol) in dry acetone at 70° C. The reaction mixture was stirred for 20 h under nitrogen. After the end of the reaction, the crude reaction mixture was filtered, and the product was then isolated by column chromatography with THF/n-hexane on a silica gel. Further purification was performed by recrystallization from THF/n-hexane. Yield 75%.

### Example 7

### Synthesis of Diblock Copolymers of PEG and P(2-VBOPNA) (PEG-b-P(2-VBOPNA))

The block copolymer, PEG₅₀₀₀-b-P(2-VBOPNA)₂₀₇₀, as a representative example, was synthesized by the following procedure: The PEG₅₀₀₀-Br macroinitiator (0.2 g, 0.04 mmol), 2-VBOPNA (0.109 g, 0.32 mmol), and Cu(I)Br (0.023 g, 0.16 mmol) were added to a flame-dried round-bottom flak. The flask was evacuated and refilled with dry nitrogen twice. Toluene (1 mL) was degassed separately and added into the flask. After the mixture was stirred and purged with N₂ for 10 min, PMDETA (0.027 g, 0.16 mmol) was introduced and the flask was placed in a preheated oil bath. The reaction was maintained at 80° C. for 2 h. The reaction solution became gradually more viscous. After the polymerization, the reaction mixture was diluted with methylene chloride and passed through a silica gel column to remove the copper catalyst. The block copolymers were purified by the repeated precipitation from methylene chloride into diethyl ether. Further purification was performed by dissolving block copolymers in water at 50° C., followed by filtration with a 0.2 μm nylon filter to remove the possible P(2-VBOPNA) homopolymer and freeze-drying. Another block copolymer, MPEG₅₀₀₀-b-P(2-VBOPNA)₁₀₄₀, which has a different chain length of (P2-VBOPNA) was synthesized in an identical manner except that a different feed molar ratio of 2-VBOPNA to EG unit of PEG₅₀₀₀-Br was employed. PEG₅₀₀₀-b-P(2-VBOPNA)₂₀₇₀: Yield 72%,

### Example 8

### Synthesis of Methyl-poly(ethylene glycol)-block-poly(2-(4-vinylbenzyloxy)-nicotinamide)

The overall synthetic route for poly(ethylene glycol)-block-poly(2-(4-vinylbenzyloxy)-nicotinamide)) is shown below. The diblock copolymer was prepared by similar methods used for the synthesis of PEG-b-P(2-VBOPNA) and PEG-b-P(2-VBODENA). Instead of using 2-VBOPNA or 2-VBODENA, 2-VBONA was used for block copolymerization.

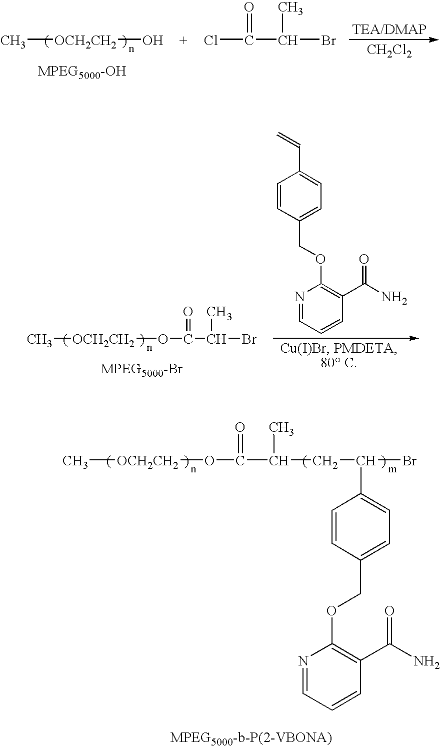

### Example 9

### Synthesis of Poly(oligoethylene glycol methacrylate-co-poly(2-(4-vinylbenzyloxy)-N,N-diethylnicotinamide)

The synthesis of a copolymer having a plurality of PEG grafts along a block of a methacrylate polymer backbone, as well as a plurality of hydrotropic agents appended to a second block of the backbone, is exemplified. Poly(oligoethyleneglycol methacrylate-co-poly(2-(4-vinylbenzyloxy)-N,N-diethylnicotinamide) is a model copolymer having the PEG block as a “graft” to a hydrotropic polymer backbone having pendant N,N-diethylnicotinamide groups. It should be noted, however, that in this scheme the PEG “graft” is actually formed by random copolymerization of a vinyl PEG derivative with a hydrotropic monomer unit, rather than by grafting the PEG moiety to a preformed polymer backbone.

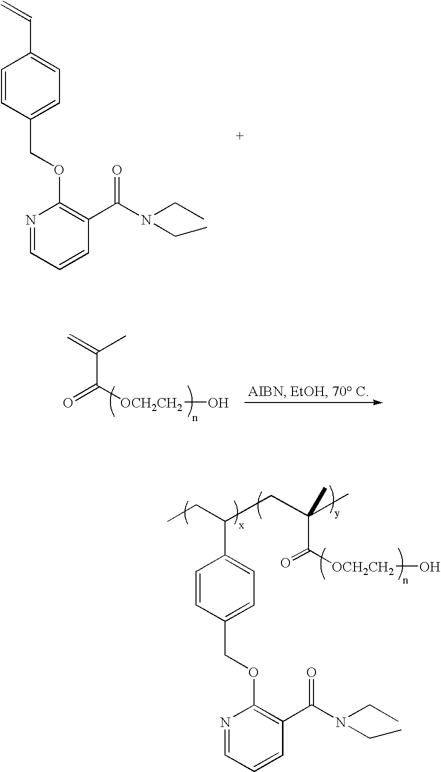

To a solution of 2-VBODENA (5 g, 0.016 mol) and oligoethylene glycol methacrylate (5.2 g, 0.18 mol) in ethanol, AIBN as an initiator (0.3 g, 3 mol % to monomer) was added. The reaction mixture was degassed with a stream of nitrogen for 30 min and polymerization was carried out at 70° C. for 24 h. After the reaction, the solution was poured into n-hexane to obtain the precipitates of the copolymer, followed by drying in vacuo at 60° C. for 24 h. In the formula, x and y depend on the relative monomer ratios, the feed weight of the monomers, and the polymerization conditions. Typically, the x:y ratio is in the range of 1:10 to 1:1 to 10:1 and the number of monomer units in the copolymer (x+y) is 10 to 100,000, preferably 200-1000.

### Example 10

### Synthesis of Poly(oligoethylene glycol methacrylate-co-poly(2-(4-vinylbenzyloxy)-N-picolylnicotinamide)

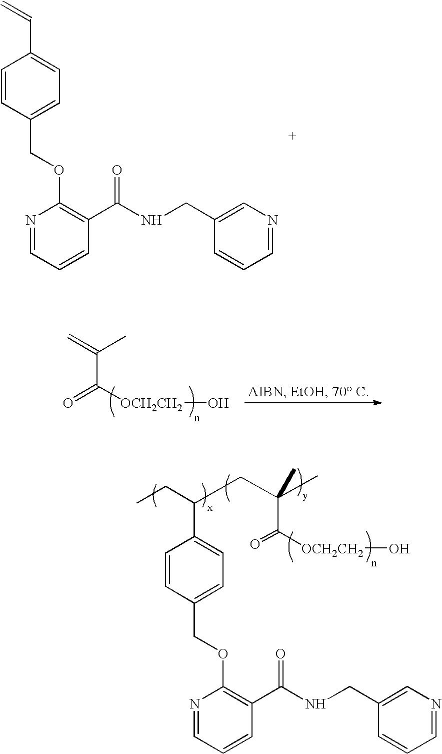

To a solution of 2-VBOPNA (5 g, 0.014 mol) and oligoethylene glycol methacrylate (4.6 g, 0.014 mol) in ethanol, AIBN as an initiator (0.25 g, 3 mol % to monomer) was added. The reaction mixture was degassed with a stream of nitrogen for 30 min. The polymerization was carried out at 70° C. for 24 h. After the reaction, the solution was poured into n-hexane to obtain the precipitates of the random copolymer, followed by drying in vacuo at 60° C. for 24 h.

### Example 11

### Synthesis of Poly(oligoethyleneglycol methacrylate-co-poly(2-(4-vinylbenzyloxy)-nicotinamide)

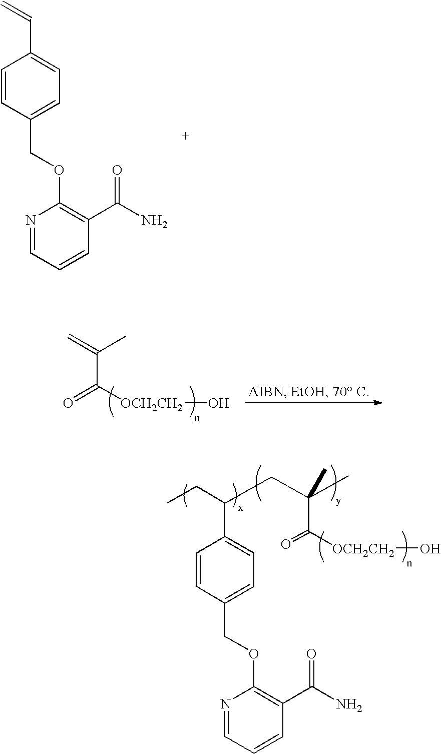

To a solution of 2-VBONA (5 g, 0.02 mol) and oligoethylene glycol methacrylate (7 g, 0.02 mol) in ethanol, AIBN as an initiator (0.35 g, 3 mol % to monomer) was added. The reaction mixture was degassed with a stream of nitrogen for 30 min. The polymerization was carried out at 70° C. for 24 h. After the reaction, the solution was poured into n-hexane to obtain the precipitates of the copolymer, followed by drying in vacuo at 60° C. for 24 h.

The hydrophilic PEG in the block copolymer can be replaced by other hydrophilic polymers, such as poly(acrylic acid) (PAA), poly(N-isopropylacrylamide), polyoxazoline, and poly(N-vinylpyrrolidone). The structural variation in the hydrophilic polymer can produce hydrotropic micelles with enhanced capability for effective drug absorption. Some examples follow.

### Example 12

### Synthesis of Poly(acrylic acid)-block-P(VBODENA) (PAA-b-P(VBODENA)

An example of the synthesis of a copolymer having a PAA block and the polymer backbone with the hydrotropic agents is described for poly(acrylic acid)-block-P(VBODENA) (PAA-b-P(VBODENA) as a model copolymer. Semitelechelic poly(tert-butyl acrylate) can be obtained by polymerization of tert-butyl acrylate in the presence of mercaptoethanol as chain transfer agent. tert-Butyl acrylate, mercaptoethanol and azobisisobutyronitrile (AIBN) can be dissolved in methanol with a predetermined feed ratio of monomer and chain transfer agent. The solution is bubbled with nitrogen gas for 30 min and kept at 70° C. with stirring for 24 h. Finally, the solution is concentrated and precipitated with excessive cold diethyl ether. Semitelechelic poly(tert-butyl acrylate) having different molecular weights can be obtained by varying the feed ratios between the chain transfer agent and monomer. The semitelechelic poly(tert-butyl acrylate) can be used as a macroinitiator for atomic radical transfer polymerization of modified hydrotropic monomers after modification with 2-bromopropionyl bromide. A series of block copolymers with different block lengths and compositions can be synthesized in controlled fashion. PAA-b-P(VBODENA), a final polymer, can be obtained by deprotection of tert-butyl units in poly(tert-butyl acrylate) with trifluoroacetic acid. The reaction is illustrated schematically for a generic hydrotropic monomer (HT).

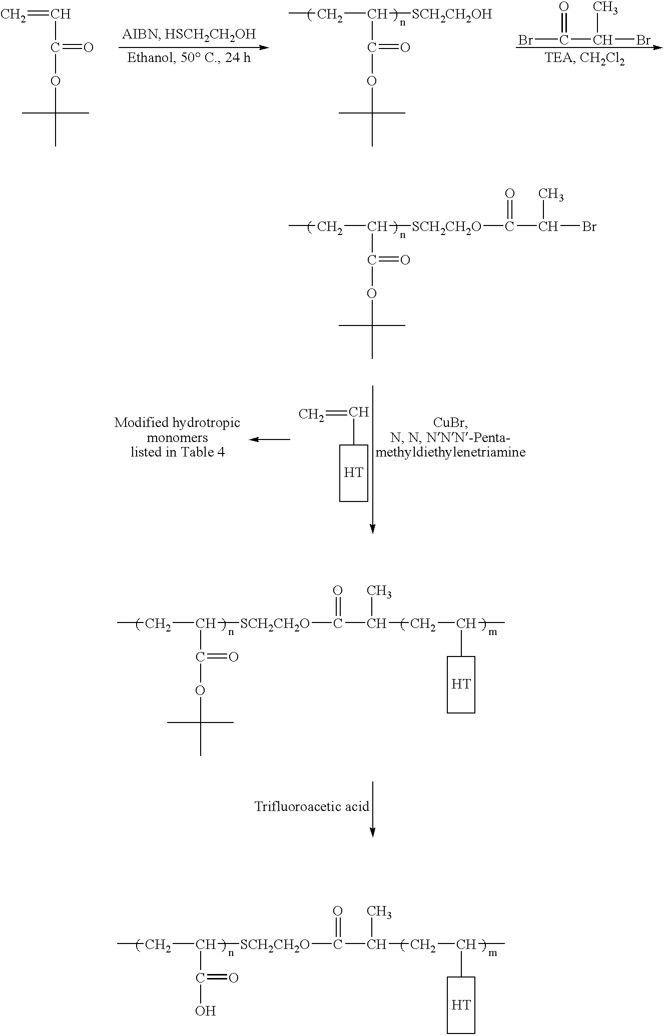

### Example 13

### Synthesis of Poly(N-isopropylacrylamide)-block-P(VBODENA) (PNIPAm-b-P(VBODENA)

PNIPAm-b-P(VBODENA can be synthesized by the reaction scheme illustrated below. Semitelechelic PNIPAAm homo- or co-polymers can be synthesized by (co)polymerization of NIPAAm and other monomers in the presence of mercaptoethanol as a chain transfer agent according to the same method used for PAA-b-P(VBODENA) and atomic transfer radical polymerization of modified hydrotropic monomers.

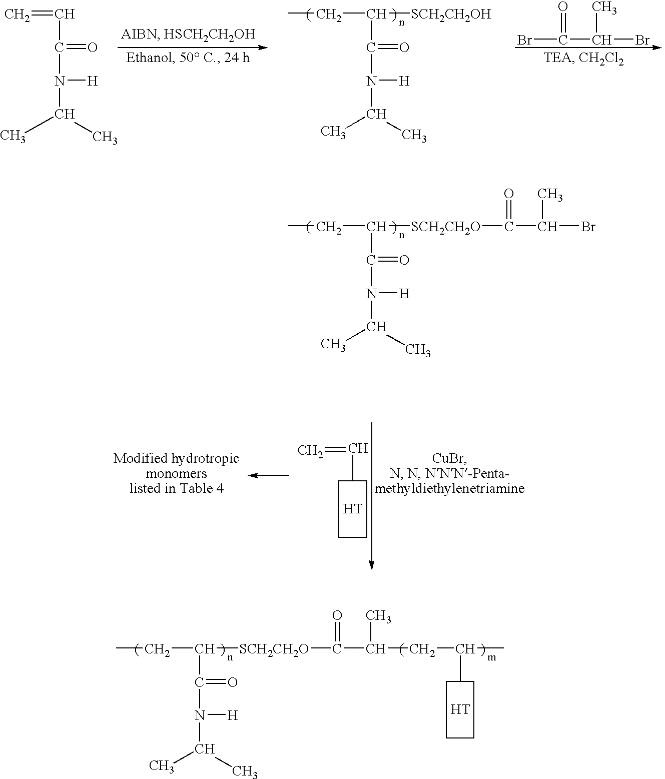

### Example 14

### Synthesis of Poly(2-ethyl-2-oxazoline)-block-P(VBODENA)(PEtOz-b-P(VBODENA)

PEtOz-b-P(VBODENA) can be synthesized by the reaction scheme illustrated below. Semitelechelic PEtOz homopolymers can be synthesized by ring-opening cationic copolymerization of 2-ethyl-2-oxazoline in the presence of methyl tosylate as an initiator. The end OH group of PEtOz can be modified with 2-bromopropionyl bromide to produce PEtOz-Br, which can be used for atom transfer radical polymerization of 2-VBODENA.

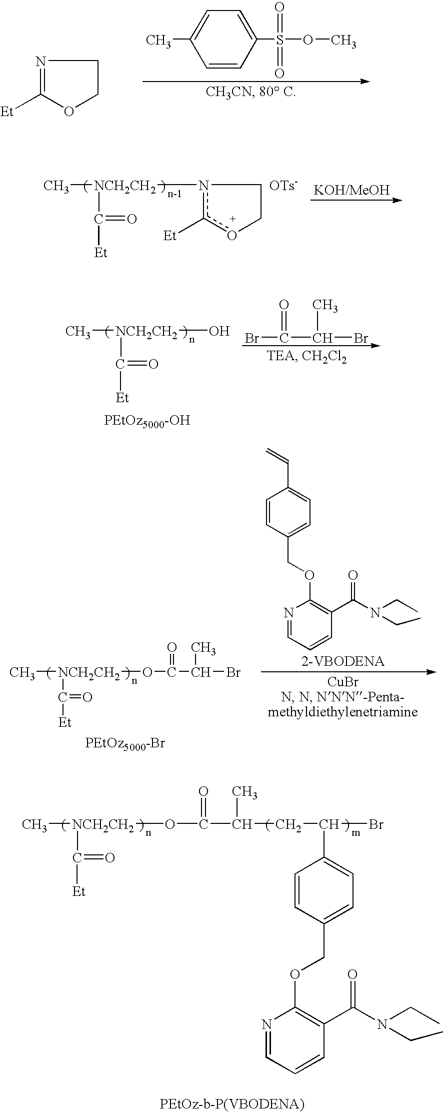

### Example 15

### Synthesis of Poly(N-vinyl pyrrolidone)-block-P(VBODENA) (PVP-b-P(VBODENA)

PVP-b-P(VBODENA can be synthesized by a reaction scheme illustrated below. Semitelechelic PVP homopolymers can be synthesized by polymerization of N-vinyl pyrrolidone in the presence of mercaptoethanol as a chain transfer agent. The end OH group of PVP can be modified with 2-bromopropionyl bromide to produce PVP with the Br end group, which can be used for atom transfer radical polymerization of 2-VBODENA.

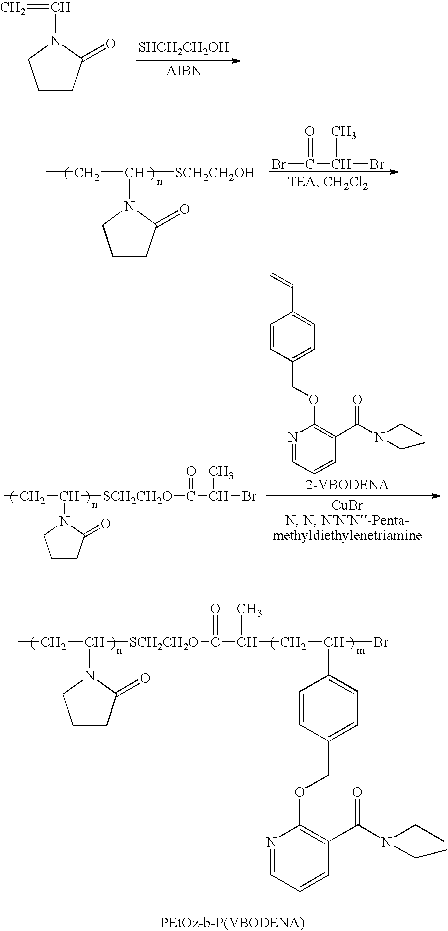

### Example 16

### Synthesis of a Series of Amphiphilic Block Copolymers with Different Hydrotropic Properties

In previous studies, the hydrotropic block copolymers consisting of hydrophilic PEG block and hydrotropic polymer block were observed to assemble in water to form polymeric micelle structures with a small size range (20˜100 nm). Hydrotrope-rich hydrophobic polymer blocks are believed to play an important role in the interaction with drug molecules. Such polymer-drug interaction may be controlled by systemically varying the chemical composition of hydrotropic blocks, making it possible to modify the solubilizing capacity and the release kinetics of hydrotropic micelles. One useful way is copolymerization of hydrotropic monomers with other hydrophilic and/or hydrophobic commoners, such as acrylic acid, acrylamide, N,N-dimethylacrylamide, N-isopropylacrylamide, butylmethacrylate, N-vinyl-2-pyrrolidinone, etc.). Various types of hydrotropic copolymers with different chemical compositions demonstrating diverse physico-chemical properties will be synthesized and investigated to find the optimum conditions for solubilization and release rate of paclitaxel. The effect of comonomers on the hydrotropic and release properties of polymeric micelles will be studied to find a useful way to control the polymer-drug interaction.

Shown is a typical example of the synthesis of a hydrotropic block copolymer. The hydrotropic moiety can be any monomers that have different affinity to paclitaxel. In the example, acrylic acid was used as a co-monomer. In this particular example, the acrylic acid moiety functions not only to decrease the affinity of the hydrotropic block to paclitaxel, but also to trigger the breakup of the hydrotropic core under neutral pH in the intestine. At neutral pH, the acrylic acid moiety becomes ionized and thus the charge repulsion will expand the hydrotropic core. At sufficiently high concentration of the acrylic acid, the core will break up at neutral pH to release paclitaxel fast. Other co-monomer such as oligo(ethylene glycol) acrylate can be added to disrupt the stacking of the hydrotropic moiety to decrease the affinity to paclitaxel.

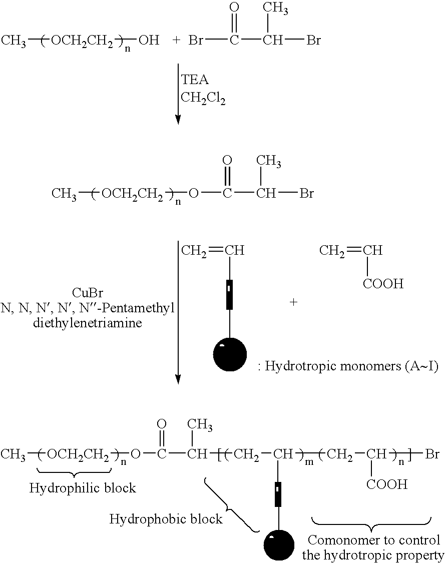

### Example 17

### Synthesis of Temperature-Sensitive Hydrotropic Block Polymers

Thermosensitive polymers are expected to be highly useful for increasing the GI transit time by interacting with the mucus layer through hydrophobic interaction as well as through increasing the viscosity or forming a gel at the body temperature. For example, poly(N-isopropylacrylamide) (PNIPAAm) nanoparticles were observed to have slower GI transit rate by enhanced adhesion to the GI tract by hydrophobic interaction than other hydrophilic and ionically interacting nanoparticles. Such thermosensitive property can be introduced to hydrotropic micellar systems to increase the transit time and therefore enhance the interaction between drug carrier and GI mucosa.

PNIPAAm is a representative thermosensitive polymer, of which lower critical solution temperature (32° C.) can be easily modulated by copolymerization with hydrophilic or hydrophobic monomers. Several diblock copolymers consisting of hydrotropic polymer blocks and thermosensitive polymers, PNIPAAm homo- and copolymers, will be synthesized by a series of synthetic procedures. Shown is a general reaction scheme of PNIPAAm-PVBODENA diblock copolymers. Semitelechelic PNIPAAm homo- or co-polymers will be synthesized by (co)polymerization of NIPAAm and other monomers in the presence of mercaptoethanol as chain transfer agent according to the same method to the previously described and used for atomic radical polymerization of hydrotropic monomers. Thermosensitive properties of the resulting block copolymers will be investigated along with micellar characterization. The proposed mechanism of enhanced GI transit time leading to improved bioavailability by the thermo-sensitive PNIPAAm-PVBODENA micelles is described in FIG. 12-b. Upon increase in temperature to 37° C., the hydrotropic polymer micelles are expected to become aggregated and also to entangle with the mucin molecules, thereby increasing the GI transit time.

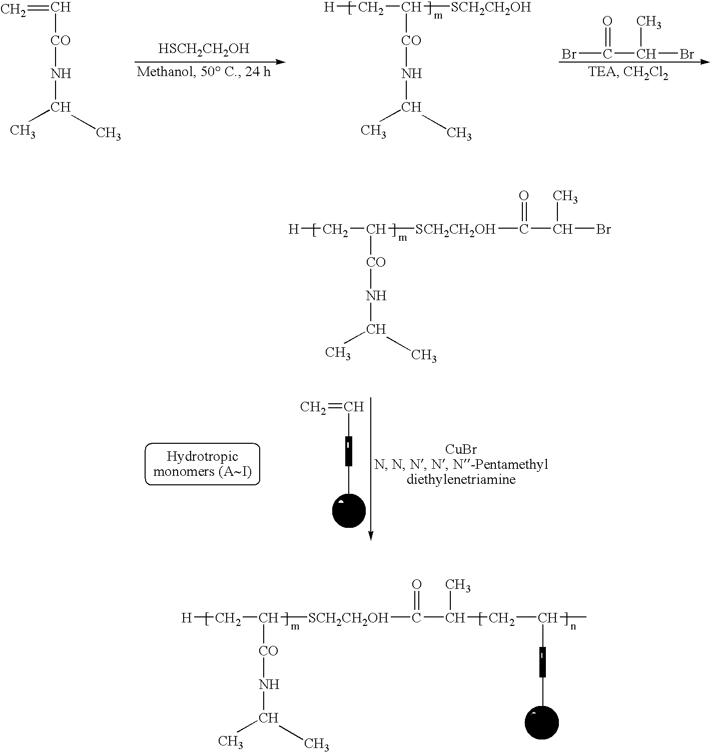

### Example 18

### Synthesis of Thermosensitive A-B-A Triblock Copolymers

Several A-B-A or B-A-B type triblock copolymers with a balanced hydrophilic and hydrophobic property, such as PLA-PEG-PLA, are well known to exhibit a thermosensitive sol-gel transition. Hydrotropic polymer blocks will be investigated as a hydrophobic block for thermosensitive polymer systems. Several kinds of triblock copolymers consisting of hydrophilic PEG blocks and hydrotropic polymer blocks will be synthesized to find an optimized block structure to demonstrate a sol-gel transition property. The triblock copolymers can be synthesized by atomic radical polymerization of hydrotropic monomers using bifunctional PEG macroinitiator. The block lengths of hydrophilic and hydrophobic parts can be controlled and optimized to maximize the solubilizing capacity and obtain the desirable polymer properties for oral delivery.

### B. Synthesis of Hydrotropic Polymer Micelles with Different Affinities to Paclitaxel

The paclitaxel affinity of hydrotropic polymer micelles can be varied by varying the hydrotropic moiety, spacer, and hydrotropic block length, and incorporating hydrophilic monomers to the hydrotropic block. The hydrophilic PEG block can be replaced with the mucoadhesive block based on poly(acrylic acid) (PAA) for long-term retention in the GI tract. In addition to the mucoadhesive block, inverse thermosensitive polymer block based on N-isopropylacrylamide can be used to increase the GI transit time by forming gels at 37° C. The volume transition temperature of N-isopropylacrylamide is around 30° C. and so it is ideal for making the polymeric micelle systems that can form gels at the body temperature.

Previous studies focused on the synthesis of simple polymeric structures, such as homopolymer and di-block copolymers, with hydrotropic properties. Polymeric and supramolecular structures based on low molecular weight hydrotropic agents have been shown to maintain the hydrotropic property. The water-solubility of paclitaxel was significantly increased by several orders of magnitude. The studies proposed focus on the optimization of polymeric structures for highly effective solubilization as well as for the ability to control the release kinetics.

To control the paclitaxel-solubilizing capacity of hydrotropic polymers, copolymers with other functional monomers, including hydrophilic, hydrophobic, thermosensitive, pH-sensitive monomers, can be synthesized. The formulations with the highest paclitaxel affinity are not necessarily the best for oral delivery. The higher stability means slower release, which is not desirable for oral delivery where the GI transit time is limited to several hours. For this reason, the formulations that can release paclitaxel fast to the surrounding medium are critical in developing oral formulations. The affinity to paclitaxel by hydrotropic polymers ca be adjusted by making copolymers using various monomers, as described herein.

### C. Solubilization Capability of the Hydrotropic Polymer Micelles

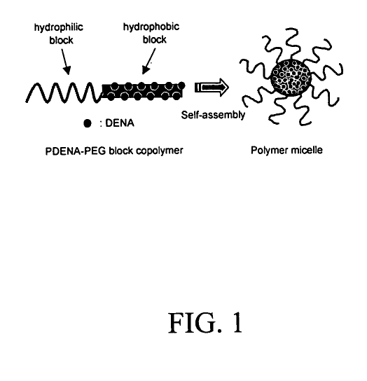

The solubilizing (loading)-effect of hydrotropic polymer micelles was tested by the dialysis method (30) and the results are listed in Table 5 and illustrated in FIG. 1. The hydrotropic polymeric micelles solubilized paclitaxel at a level of 18.4-37.4 wt %, depending on the organic solvents used in the dialysis and the initial feed weight ratio of paclitaxel to the block copolymer. The loading content increases to certain feed weight ratios of paclitaxel to the block copolymer. However, as the amount of paclitaxel increased further, precipitates of unloaded paclitaxel were formed during dialysis, resulting in decreased loading contents. The maximum loading was observed with initial feed weight ratio of 1:5.0. Especially, when acetonitrile and the feed weight ratio of 1:5. were used, the loading content was as high as 37.4 wt %, which was not possible with existing polymeric micelle systems. Dimethylformamide (DMF) and dimethylacetamide (DMAc) were also studied. The maximum loading content of paclitaxel in a control micelle of PEG₂₀₀₀-PDLLA₂₀₀₀ (30) was estimated to be 27.6 wt %, which is close to the literature value.

TABLE 5
Paclitaxel loading contents in PEG₅₀₀₀-b-P(VBODENA)₄₃₅₀ micelles

| Feed weight ratio | | |
|---|---|---|
| (Polymer: Paclitaxel) | Solvent | Loading content (wt %) |
| 1:0.25 | DMF | 14.5 |
| 1:3.0 | | 19.5 |
| 1:3.5 | | 22.0 |
| 1:4.0 | | 28.2 |
| 1:4.5 | | 30.2 |
| 1:5.0 | | 33.0 |
| 1:6.0 | | 31.5 |
| 1:2.5 | CH₃CN | 18.4 |
| 1:3.0 | | 22.8 |
| 1:3.5 | | 28.7 |
| 1:4.0 | | 30.1 |
| 1:4.5 | | 33.2 |
| 1:5.0 | | 37.4 |
| 1:6.0 | | 33.3 |
| 1:2.5 | DMAc | 18.8 |
| 1:3.0 | | 20.9 |
| 1:3.5 | | 26.4 |
| 1:4.0 | | 27.9 |
| 1:4.5 | | 19.2 |
| 1:5.0 | | 31.2 |
| 1:6.0 | | 29.0 |

The data in Table 6 also show that the loading capacity of PEG-b-P(VBODENA) micelles for paclitaxel was enhanced with increasing block length of the P(VBODENA) polymer block.

TABLE 6
Paclitaxel loading contents in PEG₅₀₀₀-b-P(VBOPNA)₂₀₇₀ micelles

| Feed weight ratio | | |
|---|---|---|
| (Polymer: PTX) | Solvent | Loading content (wt %) |
| 1:0.25 | DMF | 15.3 |
| 1:3.0 | | 19.5 |
| 1:3.5 | | 25.3 |
| 1:4.0 | | 30.1 |
| 1:4.5 | | 31.2 |
| 1:5.0 | | 33.2 |
| 1:6.0 | | 28.2 |

Paclitaxel-loaded micelles were freeze-dried and could be redissolved as micelles by a simple vortexing and heating at 60° C. for 1 min to give a wide range of paclitaxel concentration. As an example, micelles containing 25.9 wt % of paclitaxel could be dissolved in water with a concentration up to 15 wt %, which corresponded to paclitaxel solubility of 38.9 mg/mL. This is about 130,000-fold increase in water solubility of paclitaxel, compared with its intrinsic water-solubility (0.3 μg/mL).

### C. Stability of Paclitaxel-Loaded Hydrotropic Polymer Micelles

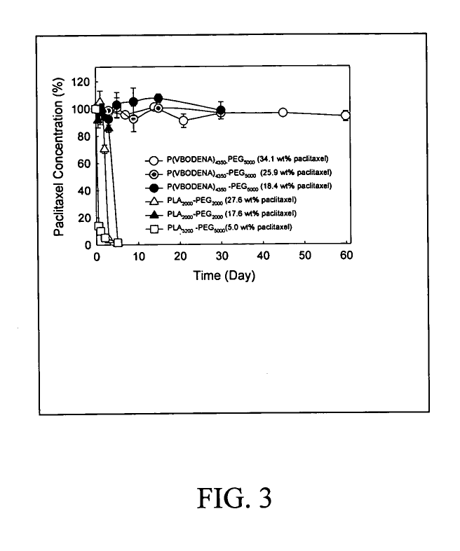

The physical stability of paclitaxel-loaded micelles was studied at 25° C. using different loading contents of paclitaxel. FIG. 3 shows time-dependent changes of the paclitaxel concentration in micelles. It is notable that the paclitaxel concentrations in P(VBODENA)-PEG micelles were maintained for months, irrespective of the loading contents. The micelle with 34.1 wt % loading was observed for long-term stability and showed no significant change in paclitaxel concentration for more than two months. On the other hand, the paclitaxel concentrations in PLA-PEG micelles were dramatically decreased only after 1˜3 days due to the precipitation of paclitaxel. Furthermore, the stability of PLA-PEG micelles became even lower as the loading content of paclitaxel increased to 27.6 wt % which was the maximum that could be achieved with the system. It lost 30% of the initial paclitaxel concentration after 2 days and retained only 4% after 3 days. PLA₃₂₀₀-PEG₅₀₀₀ micelles showed much lower stability even at a much lower drug loading. The good stability of paclitaxel-loaded PEG-b-P(VBODENA) micelles was also confirmed by dynamic light scattering. No appreciable change in micelle sizes was observed at 25° C. for weeks and the micelle diameters of about 105˜120 nm were maintained for more than 8 weeks.

### II. Preparation and Evaluation of Paclitaxel Formulations

### A. Preparation of Paclitaxel/Hydrotropic Polymer Micelle Formulations

### 1. Current Commercial Paclitaxel Formulation

Paclitaxel is clinically proven active against advanced ovarian and breast cancer and is under investigation for various other types of cancers. The recommended doses for clinical applications of paclitaxel are 135 mg/m² and 175 mg/m² for small (1.4 m²) and large (2.4 m²) patients, respectively. These equal the total paclitaxel quantities of 189 mg and 420 mg. The current clinical dosage form of paclitaxel consists of a 5 ml vial containing a total of 30 mg of paclitaxel, 2.635 g of Cremophor EL, and 49.7% ethanol (1:1 v/v), which is to be diluted with 0.9% sodium chloride or 5% dextrose injection solution to 0.3 mg/ml or 1.2 mg/ml before i.v. administration. Even with the use of Cremophor and ethanol, the total volume of the delivery solution is either 350 ml or 630 ml. If one uses pure water, then the delivery volumes would increase to 630 liters and 1,400 liters, which are physically impossible to deliver. The poor solubility has resulted in serious formulation problems, and this has also caused difficulties in other routes of delivery, such as oral administration. The presence of hydrotropic polymers is expected to eliminate the use of Cremophor EL, and ethanol in the paclitaxel formulation, lowering the toxicity of the current formulation significantly. The oral paclitaxel formulations using hydrotropic polymers are expected -to increase the paclitaxel bioavailability due to the increased paclitaxel solubility in water.

### 2. Paclitaxel/Hydrotropic Polymer Micelle Formulations

The minimum effective concentration of paclitaxel is known to be 0.1 μmol/L, which is equivalent to approximately 0.1 μg/ml (0.1 μmol/L×854 g/mol=0.0854 μg/ml˜0.1 μg/ml). The oral dose of the paclitaxel/hydrotropic polymer micelle formulations are adjusted to obtain the blood paclitaxel concentration of 0.1 μg/ml and higher. A recent study done on oral administration of water-soluble paclitaxel derivatives used the oral dose of paclitaxel derivatives varying from 50 mg/kg to 200 mg/kg. Thus, the similar range of paclitaxel is employed in the beginning. The i.v. dose is varied from 10 mg/kg to 50 mg/kg.

The paclitaxel formulations are based on hydrotropic polymer micelles, which, due to their large molecular weights, are not absorbed from the GI tract and remain on the surface of the GI tract to provide a continuous supply of paclitaxel.

The liquid formulations are prepared by dissolving paclitaxel-loaded hydrotropic polymer micelles in aqueous solution first to the desired concentrations. The liquid formulations are administered to rats through chronically implanted catheters, as described hereinbelow. The presence of chronic catheters allows administration of liquid dosage form, and the effect of a hydrotropic polymer formulation can be tested easily. This particular approach is useful since the administered hydrotropic polymer micelle solution is not diluted much by the fluid present in the GI tract of the rats. Thus, the effect of high paclitaxel solubility in aqueous solution (1˜10 mg/ml and higher) on bioavailability can be tested. All aqueous solutions are prepared just before use.

### B. Cytotoxicity Evaluation of Hydrotropic Polymer Micelle Formulations

The antitumor cytotoxicities, as measured by ED₅₀, of polymeric micelles on various cell lines were measured as shown in Table 7. The results of cytotoxicity of hydrotropic polymer micelles and control micelles clearly show the superior cytotoxic properties of hydrotropic micelles. Free paclitaxel and doxorubicin in ethanol were used as positive controls. The ED₅₀ values of hydrotropic micelles are much lower than PLA-PEG and PPA-PEG micelles. The most widely used polymeric micelle is PLA-PEG micelles with the maximum paclitaxel loading capacity of 24 wt %. Although the hydrotropic polymer micelles have higher paclitaxel loading up to 37%, hydrotropic polymer micelles with only 20% and 25% paclitaxel loading were used to compare the efficacy with that of PLA-PEG micelles. At equivalent paclitaxel loading, i.e., 25% loading, hydrotropic polymer micelles were substantially more effective. In the case of MDA231 cell line, hydrotropic polymer micelles were more than two orders of magnitude more effective. The data clearly indicate that hydrotropic polymer micelles are not only more stable in aqueous solution, but also more effective.

TABLE 7
ED₅₀ (μg/ml) of paclitaxel and paclitaxel (PTX)-loaded
polymeric micelles on various tumor cell lines.

| | Cancer cell lines | | | |
|---|---|---|---|---|
| Samples | HT-29 | MDA231 | MCF-7 | SKOV-3 |
| Doxorubicin (positive control) | 0.044 | 0.050 | 0.773 | 0.611 |
| Paclitaxel | 0.003 | 0.033 | 0.043 | 0.006 |
| PTX-loaded HTM (25 wt % | 0.005 | 0.002 | 0.002 | 0.001 |
| loading) | | | | |
| PTX-loaded HTM (20 wt % | 0.006 | 0.004 | <0.001 | 0.008 |
| loading) | | | | |
| PTX-loaded PLA-PEG 924 | 0.014 | 0.305 | <0.001 | 0.015 |
| wt % loading) | | | | |
| PTX-loaded PPA-PEG | 0.012 | 1.077 | 0.002 | 1.543 |
| HTM alone | 4.221 | 5.650 | 5.767 | 0.048 |
| PLA-PEG alone | 4.672 | 8.435 | 5.533 | — |
| PPA-PEG alone | 0.277 | 4.881 | 6.194 | — |

PTX: paclitaxel,
HTM: hydrotropic micelle,
PLA: poly(lactic acid),
PEG: poly(ethylene glycol),
PPA: poly(phenylalanine)

The present invention has been described hereinabove with reference to particular examples for purposes of clarity and understanding rather than by way of limitation. It should be appreciated that certain improvements and modifications to the present invention can be practiced within the scope of the appended claims.

References

The pertinent portions of the following references are incorporated herein by reference:
1. Yalkowsky, S. H. Solubility and Solubilization in Aqueous Media. American Chemical Society, Washington, D.C., p. 429, 1999.
2. Löbenberg, R., et al. “Solubility as a limiting factor to drug absorption,” in Oral Drug Absorption. Prediction and Assessment. J. B. Dressman and H. Lennernäs, eds., pp. 137-153, Marcel Dekker, New York, 2000.
3. Sharma, A. et al., “Novel taxol formulations: preparation and characterization of taxol-containing liposomes,” Pharm. Res., 11: 889-896, 1994.
4. Alkan-Onyuksel, et al., “A mixed micellar formulation suitable for the parenteral administration of taxol,” Pharm. Res., 11: 206-212, 1994.
5. Rubino, J. T. et al., “Cosolvency and cosolvent polarity,” Pharm. Res., 4: 220-230, 1987.
6. Goldspiel, B. R., “Clinical overview of the taxanes,” Pharmacotherapy, 17: 110S-125S, 1997.
7. Adams, J. D., et al., “Taxol: a history of pharmaceutical development and current pharmaceutical concerns,” J. Natl. Cancer Inst. Monogr., 15: 141-147, 1993.
8. Mazzo, D. J., et al., “Compatibility of docetaxel and paclitaxel in intravenous solutions with poyvinyl chloride infusion materials,” Am. J. Health-Syst. Pharm., 54: 566-569, 1997.
9. Singla, A. K., et al., “Paclitaxel and its formulations,” Int. J. Pharm., 235: 179-192, 2002.
10. Mani, S., et al., “Phase I clinical and pharmacokinetic study of oral 9-aminocamptothecin (NSC-603071),” Cancer Chemother. Pharmacol., 42: 84-87, 1998.
11. Macheras, P., et al., Biopharmaceutics of orally administered drugs. Ellis Horwood, New York, 1995.
12. Allen, C., et al., “Nano-engineering block copolymer aggregates for drug delivery,” Colloids Surf. B: Biointerfaces, 16: 3-27, 1999.
13. Kabanov, A. V., et al., “A new class of drug carriers: micelles of poly(oxyethylene)-poly(oxypropylene) block copolymers as microcontainers for-drug targeting from blood in brain,” J. Control. Release, 22: 141-158, 1992.
14. Kataoka, K., et al., “Block copolymer micelles as vehicles for drug delivery,” J. Control. Release, 24: 119-132, 1993.
15. Kwon, G. S., et al., “Physical entrapment of adriamycin in ab block copolymer micelles,” Pharm. Res., 12: 192-195, 1995.
16. Allen, C., et al., “Polycaprolactone-b-poly(ethylene oxide) copolymer micelles as a delivery vehicle for dihydrotestosterone,” J. Control. Release, 63: 275-286, 2000.
17. Kim, S. Y., et al., “Toxic characteristics of methoxy poly(ethylene glycol)/poly(e-caprolactone) nanospheres; in vitro and in vivo studies in the normal mice,” Biomaterials, 24: 55-63, 2003.
18. Inoue, T., et al., “An AB block copolymer of oligo(methyl methacrylate) and poly(acrylic acid) for micellar delivery of hydrophobic drugs,” J. Control. Release, 51: 221-229, 1998.
19. Burt, H. M., et al., “Development of copolymers of poly(D,L-lactide) and methoxypolyethylene glycol as micellar carriers of paclitaxel,” Colloids Surf. B: Biointerfaces, 16: 161-171, 1999.
20. Kim, S. C., et al., “In vivo evaluation of polymeric micellar paclitaxel formulation: toxicity and efficacy,” J. Control. Release, 72: 191-202, 2001.
21. Lee, S. C., et al., “Polymeric micelles of poly(2-ethyl-2-oxazoline)-block-poly(e-caprolactone) copolymer as a carrier for paclitaxel,” J. Controlled Release, 89: 437-446, 2003.
22. Coffman, R. E. et al. “Effect of nicotinamide and urea on the solubility of riboflavin in various solvents,” J. Pharm. Sci., 85: 951-954, 1996.
23. Saleh, A. M., et al., “Solubilization of water by hydrotropic salts,” J. Pharm. Sci., 75: 644-647, 1986.
24. Dhara, D., et al. “Effect of hydrotropes on the volume phase transition in poly(N-isopropylacrylamide) hydrogel,” Langmuir, 15: 930-935, 1999.
25. Srinivas, V. et al., “When does the switch from hydrotropy to micellar behavior occur?” Langmuir, 14:,6658-6661, 1998.
26. Kumar, M. D., et al., “ffect of hydrotropes on solubility and mass transfer coefficient of methyl salicylate,” Chem. Eng. Data, 45: 419-423, 2000.
27. Lee, J., et al., “Hydrotropic solubilization of paclitaxel: analysis of chemical structures for hydrotropic property,” Pharm. Res., 20: 1022-1030, 2003.
28. Lee, S. C., et al., “Hydrotropic polymers: synthesis and characterization of polymers containing picolylnicotinamide moieties,” Macromolecules, 36: 2248-2255, 2003.
29. Vasilescu, M., et al., “Aggregation number and microstructure characterization of self-assembled aggregates of poly(ethylene oxide) surfactants and related block-copolymers, studied by spectroscopic methods,” Advances in Colloid and Interface Science, 89-90: 169-194, 2001.
30. Huh, K. M., et al., “Hydrotropic polymer micelle system for delivery of paclitaxel,” J. Controlled Rel., in press.

1. A copolymer effective for increasing aqueous solubility of a poorly soluble drug, which copolymer comprises a plurality of at least one hydrophilic monomer unit and a plurality of at least one hydrophobic monomer unit, wherein the hydrophobic monomer unit possesses a pendant hydrotropic moiety.

2. The copolymer of claim 1, wherein the at least one hydrophilic monomer is selected from the group consisting of ethylene glycol, oligoethylene glycol methacrylate, acrylic acid, methacrylic acid, N-isopropylacrylamide, N-vinylpyrrolidone, 2-methyl-2-oxazoline, and 2-ethyl-2-oxazoline.

3. The copolymer of claim 1, wherein the plurality of hydrophilic monomer units is present in the copolymer as a hydrophilic polymer block.

4. The copolymer of claim 3, wherein the hydrophilic polymer block is comprised of polyethyleneoxide.

5. The copolymer of claim 1, wherein the at least one hydrophobic monomer unit is selected from the group consisting of polymerizable derivatives of nicotinamide or salicylate.

6. The copolymer of claim 5, wherein the hydrophobic monomer unit is an acryl or styryl derivative of nicotinamide or salicylate.

7. The copolymer of claim 6, wherein the hydrophobic monomer unit is an acryl or styryl derivative of an N-substituted nicotinamide selected from the group consisting of N,N-diethylnicotinamide, N-picolylnicotinamide, N-allylnicotinamide, N,N-dimethylnicotinamide, and N-methylnicotinamide.

8. The copolymer of claim 1, wherein the plurality of at least one hydrophobic monomer units is present in the copolymer as a hydrophobic polymer block.

9. The copolymer of claim 1 in the form of a tri-block, random or graft polymer.

10. The copolymer of claim 1, which is effective in increasing water solubility of paclitaxel by at least a factor of 100.

11. A method for making the copolymer of claim 1, comprising reacting a hydrophilic homopolymer and a plurality of polymerizable hydrophobic monomer units in the presence of a polymerization catalyst.

12. The method of claim 11, wherein the polymerization catalyst comprises a metal halide and an amine ligand.

13. The method of claim 12, wherein the metal halide is CuCl or CuBr.

14. The method of claim 12, wherein the amine ligand is a copper-complexing compound selected from 2,2′-dipyridyl, copper-complexing derivatives of 2,2′-dipyridyl, N,N,N′,N′,N″-pentamethyldiethylenetriamine, N,N,N′,N″,N″-hexamethyltriethylenetetramine, and tris[(2-dimethylamino)ethyl]amine.

15. A pharmaceutical composition comprising a plurality of hydrotropic polymer micelles loaded with a pharmacologically effective amount of a poorly soluble drug, wherein the micelles are comprised of an amphiphilic copolymer formed of a plurality of at least one hydrophilic monomer unit and a plurality of at least one hydrophobic monomer unit possessing a pendant hydrotropic moiety.

16. The composition of claim 15, wherein the plurality of at least one hydrophilic monomer unit is present in the copolymer in the form of poly(ethylene glycol), poly(oligoethylene glycol methacrylate), poly(acrylic acid), poly(methacrylic acid), poly(N-isopropylacrylaamide), poly(N-vinylpyrrolidone), poly(2-methyl-2-oxazoline), or poly(2-ethyl-2-oxazoline).

17. The composition of claim 15, wherein the plurality of at least one hydrophobic monomer unit is present in the copolymer in the form of a block of polymerizable derivatives of nicotinamide and N-substituted nicotinamide.

18. The composition of claim 17, wherein the N-substituted nicotinamide is selected from the group consisting of N,N-diethylnicotinamide, N-picolylnicotinamide, N-allylnicotinamide, N,N-dimethylnicotinamide, and N-methylnicotinamide.

19. The composition of claim 15, wherein the copolymer is in the form of a tri-block, random or graft copolymer.

20. The composition of claim 15, wherein the poorly soluble drug is paclitaxel.

21. A method of increasing water solubility of a hydrophobic compound comprising combining said hydrophobic compound with a hydrotropic polymer micelle formed from the copolymer of claim 1.

22. A method of treating a patient with a drug comprising co-administering the drug and a hydrotropic polymer micelle to the patient.

23. The method of claim 22, wherein the drug is loaded inside the hydrotropic polymer micelle prior to administration to the patient.

24. The method of claim 22, wherein the drug and the hydrotropic polymer micelle are administered orally or intravenously.

25. A method of forming a solid dispersion of a hydrophobic drug and a hydrotropic polymer micelle comprising melting the drug in the presence of a hydrotropic agent, copolymer or hydrogel, and allowing the resulting composition to cool.

26. The method of claim 25, wherein the drug is paclitaxel.

27. The method of claim 25, wherein the micelle is formed by an amphiphilic diblock copolymer.

28. A method of forming a liquid dispersion of nanoparticles composed of a hydrophobic drug and a hydrotropic polymer micelle comprising combining the drug and the micelle to form an admixture thereof, and contacting the admixture with water so that the nanoparticle dispersion is formed.

29. The method of claim 28, wherein the drug is paclitaxel.

30. The method of claim 28, wherein the micelle is formed by an amphiphilic diblock copolymer.
